(19) United States
(12) Patent Application Publication
(10) Pub. No.: US 2018/0263881 A1
(43) Pub. Date: Sep. 20, 2018

(54) AGENT FOR DECOLORIZING DYED KERATINOUS FIBERS

(71) Applicant: Henkel AG & Co. KGaA, Duesseldorf (DE)

(72) Inventors: Juergen Schoepgens, Schwalmtal (DE); Torsten Lechner, Langenfeld (DE); Antje Gebert-Schwarzwaelder, Neuss (DE)

(73) Assignee: Henkel AG & Co. KGaA, Duesseldorf (DE)

(21) Appl. No.: 15/920,865

(22) Filed: Mar. 14, 2018

(30) Foreign Application Priority Data
Mar. 15, 2017 (DE) 102017204290.7

(51) Int. Cl. A61K 8/46; A61Q 5/08; A61K 8/41

(52) U.S. Cl. CPC A61K 8/46; A61K 2800/882; A61K 8/41; A61Q 5/08

(57) ABSTRACT

The subject matter of the present disclosure is an agent for the reductive decolorization of dyed keratinous fibers, more particularly human hair, containing, in a cosmetic carrier:
(a) Formamidine sulfinic acid and/or salts thereof and
(a) At least one compound of formula (I)

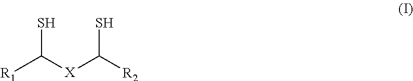

Additional subjects of the present disclosure are corresponding multi-component package units and methods for reductive decolorizing of dyed keratinous fibers.

### CROSS-REFERENCE TO RELATED APPLICATION

This application claims priority to German Patent Application No. 10 2017 204 290.7, filed Mar. 15, 2017, which is incorporated herein by reference in its entirety.

### TECHNICAL FIELD

The present disclosure relates to the cosmetic sector. The subject of the present disclosure is an agent for reductive decolorization of dyed keratinous fibers, particularly human hair, which contains formamidine sulfinic acid and/or salts thereof in a cosmetic carrier (a) and (b) a special disulfide compound of formula (I).

### BACKGROUND

Additional subjects of the present disclosure are multi-component package units which are packaged in different ways containing (a) formamidine sulfinic acid and/or salts thereof and (b) at least one disulfide compound of formula (I).

A further subject of the present disclosure is a method for reductive decolorization of dyed keratinous fibers, with which the decolorizing agent or the multi-component package unit described above is used.

Preparations for tinting and coloring hair are an important type of cosmetic agent. They can serve to tint the natural hair color to a lesser or greater degree depending on the preferences of each and every person, achieve a completely different hair color or cover unwanted color shades, such as shades of gray, for example. Routine hair dyes are formulated either on the basis of oxidation dyes or on the basis of partially-oxidizing dyes, depending on the preferred color and/or permanency of the dye. To achieve special tints, combinations of oxidation dyes and partially-oxidizing dyes are also frequently used.

Dyes formulated on the basis of oxidation dyes lead to brilliant and permanent color shades. However, they do require the use of strong oxidants, such as hydrogen peroxide solutions, for example. Said dyes contain oxidative dye precursors, so-called developer components and coupler components. The developer components join together or couple with one or more coupler components to form, under the influence of oxidants or atmospheric oxygen, the actual colorants per se.

Dyes formulated on the basis of partially-oxidizing dyes are often used for achieving temporary colors. Partially-oxidizing dyes are dye molecules that coat the hair itself and do not require an oxidative process to create the color. Important representatives of this dye class include triphenylmethane dyes, azo dyes, anthraquinone dyes or nitrobenzene dyes, each of which can carry cationic or anionic groups.

With all said dyeing methods, however, the color may need to be reversed, either whole or in part, for various reasons. A partial removal of the color may be the ideal solution, for example, if the color result has a darker effect on the fibers than desired. On the other hand, a complete removal of the color may be desired in some cases. It is conceivable, for example, that the hair is to be colored or tinted in a particular way for a specific occasion, and the original color is to be restored after a few days.

Technical literature also discloses decolorizing agents and methods. The oxidative post-treatment of dyed hair, by means of a routine bleaching agent for example, is a well-known method from the prior art. With this process, however, the fibers can also be damaged through the use of strong oxidants.

Moreover, reductive processes for decolorization have already been described. European Patent Application EP 1 300 136 A2 discloses, for example, a method for hair treatment, wherein the hair is colored in a first step and then reductively decolorized again in a second step. Said reductive decolorization is achieved by means of a formulation containing a dithionite salt and a surfactant. In WO 2008/055756 A2, the reductive decolorization of keratinous fibers is achieved using a mixture formed from a reducing agent and an absorption agent.

Another decolorizing agent well known from the prior art is Rongalit (hydroxymethane sulfinic acid), which is commercially available in the form of its sodium salt (sodium hydroxymethane sulfinate), for example. Rongalit has out outstanding decolorizing effect. For toxicological reasons, however, the use of said substance is avoided wherever possible.

When reductive decolorizing agents are used, the decolorization effect is achieved by reducing the dyes located on the keratinous fibers and/or hair. The reduction process usually involves converting the dyes to their reduced forms and/or leuco forms. This method involves reducing the double bonds present in the dyes, thus interrupting the chromophoric system of the dyes and converting the dye into a colorless form.

A general problem with the reductive decolorizing agents known from the prior art is that the decolorizing process normally requires very long application times. In order to achieve decolorization that is as complete as possible, the ready-to-use decolorizing agent is normally left on the dyed keratinous fibers or hair for approximately 60 minutes.

In order to be able to change to a desired hair color, the user accepts such long application times—however, these time-consuming processes are very uncomfortable and therefore undesired. The present disclosure addresses the problem of inventing new reductive decolorizing agents that enable color removal that is as complete as possible in the shortest possible exposure time.

### BRIEF SUMMARY

Agents for the reductive decolorization of dyed keratinous fibers and multi-component package unit (kit-of-parts) for reductive decolorizing of keratinous fibers are provided herein. In an embodiment, an agent for the reductive decolorization of dyed keratinous fibers includes, in a cosmetic carrier, (a) formamidine sulfinic acid and/or salts thereof and (b) at least one compound of the formula (I)

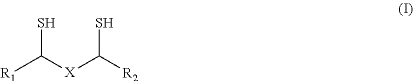

In formula (I), R1 and R2 denote independently of one another a hydrogen atom, a carboxy group (—COOH) or the salt thereof, a C₁-C₆ alkyl group, a hydroxy C₁-C₆ alkyl group, or a C₁-C₆ alkoxy-carbonyl group; X denotes a bivalent organic radical of formula (II) or a direct bond,

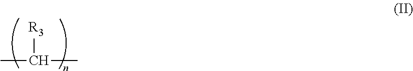

R3 denotes a hydroxy group, a hydrogen atom, a carboxy group (—COOH) or the salt thereof, a C₁-C₆ alkoxy-carbonyl group, a C₁-C₆-alkyl group or an amino group, and n denotes an integer from about 1 to about 4.

In another embodiment, a multi-component package unit (kit-of-parts) for reductive decolorizing of keratinous fibers includes, separately packaged, a first container (1) that includes a cosmetic agent (1) and a second container (2) that includes a cosmetic agent (2). Agent (1) in container (1) includes (a) Formamidine sulfinic acid and/or salts thereof and (b) at least one compound of formula (I)

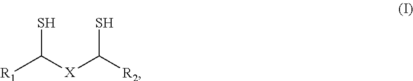

where R1, R2, X, R3, and n are as described above. Agent (2) in container (2) includes (c) one or multiple alkalizing agents.

In another embodiment, a multi-component package unit (kit-of-parts) for reductive decolorizing of keratinous fibers includes, separately packaged, a first container (1) including a cosmetic agent (1) and a second container (2) including a cosmetic agent (2). Agent (1) in container (1) includes (a) formamidine sulfinic acid and/or salts thereof. Agent (2) in container (2) includes (b) at least one compound of the formula (I),

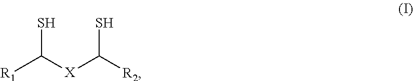

where R1, R2, X, R3, and n are as described above.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Surprisingly, it has been found that the aforementioned problem can be solved to complete satisfaction when a cosmetic agent of formula (I) containing a combination of (a) formamidine sulfinic acid (or the salts of formamidine sulfinic acid) and (b) at least one special dithio compound (or dimercapto compound) is applied on the previously dyed hair. After an exposure time of only from about 15 to about 30 minutes, the decolorizing agents, containing this combination of reducing agents (a) and (b), enabled a decolorizing effect which agents known from the prior art would have required an exposure time of from about 45 to about 60 minutes to achieve.

A first subject matter of the present disclosure is an agent reductive decolorization of dyed keratinous fibers, more particularly human hair, containing in a cosmetic carrier

(a) Formamidine sulfinic acid and/or salts thereof and
(b) At least one compound of the formula (I)

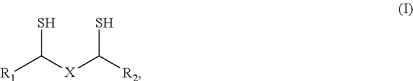

where
R1, R2 denote independently of one another a hydrogen atom, a carboxy group (—COOH) or the salt thereof, a C₁-C₆ alkyl group, a hydroxy C₁-C₆ alkyl group, or a C₁-C₆ alkoxy-carbonyl group,
X denotes a bivalent organic radical of formula (II) or a direct bond,

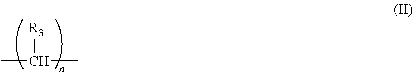

R3 denotes a hydroxy group, a hydrogen atom, a carboxy group
(—COOH) or the salt thereof, a C₁-C₆ alkoxy-carbonyl group, a
C₁-C₆-alkyl group or an amino group, and
n denotes an integer from about 1 to about 4.

The application of the decolorizing agent as contemplated herein achieves—depending on the dyes previously used for the dyeing process—an almost complete or even complete decolorization of previously dyed keratinous fibers. The decolorizing agent has an outstanding effect, above all on keratinous fibers previously dyed with oxidation dyes. In this connection it was particularly surprising to find that this decolorizing effect could already be achieved after a very should application time of approximately 15-30 minutes.

Keratinous fibers, keratin-containing fibers or keratinous fibers are furs, wool, feathers and, in particular, human hair. Although the agents as contemplated herein are most suitable for lightening and coloring keratinous fibers and/or human hair, they can in principle be used for other purposes.

The expression “dyed keratinous fibers” means keratinous fibers, which were dyed by means of conventional cosmetic dyes known to a person skilled in the art. The expression “dyed keratinous fibers” means in particular fibers that have been dyed by means of oxidative dyes and/or partially oxidizing dyes known from the prior art. In this context, explicit reference is made to the known monographies, e.g. Kh. Schrader, Grundlagen and Rezepturen der Kosmetika [Cosmetic principles and formulas, 2nd Edition, Hüthig Buch Verlag, Heidelberg, 1989, which reflect the corresponding knowledge of a person skilled in the art.

The agents for decolorization of keratinous fibers or hair contain ingredients (a) and (b), which are essential to the present disclosure, in a cosmetic carrier. This can, for example, be a suitable hydrous or hydrous-alcoholic carrier. Carriers such as creams, emulsions, gels or surfactant-containing, foaming solutions, such as shampoos, foaming aerosols, foam formulations or other preparations suitable for application on the hair, are used for the purpose of reductive decolorization. Agents for the reductive decolorization of keratinous fibers are preferably creams, emulsions or free-flowing gels.

(a) Formamidine Sulfinic Acid and/or Salts Thereof

The inventive agents contain formamidine sulfinic acid and/or salts of formamidine sulfinic acid as a first ingredient (a) that is essential to the present disclosure. Formamidine sulfinic acid is alternatively referred to as thiourea dioxide or as aminoiminomethanesulfinic acid. Formamidine sulfinic acid has the structure of formula (I), but can also be present in the form of its tautomers. Formamidine sulfinic acid has the CAS number 1758-73-2 and is commercially available from various providers, such as Sigma Aldrich.

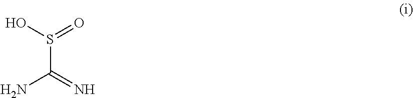

Formamidine sulfinic acid can also be used in the form of its salts. For example sodium, potassium and ammonium salts of formamidine sulfinic acid are suitable.

However, particular preference is given to formamidine sulfinic acid itself (i.e. use of the compound of formula (i)).

Formamidine sulfinic acid or salts thereof are preferably used in specific quantity ranges. Inventive agent (a) preferably contains formamidine sulfinic acid and/or salts thereof in a total amount of from about 0.1 to about 30.0 wt. %, preferably from about 1.5 to about 15.0 wt. %, more preferably from about 3.5 to about 12.0 wt. % and particularly from about 6.0 to about 10.0 wt. % All quantity specifications are relative to the total amount of formamidine sulfinic acid (salts) which are added in relation to the total weight of the agent.

In a particularly preferred embodiment, an inventive agent is exemplified in that it contains—relative to the total weight of agent—(a) formamidine sulfinic acid and/or salts thereof in a total amount of from about 0.1 to about 30.0 wt. %, preferably from about 1.5 to about 15.0 wt. %, more preferably from about 3.5 to about 12.0 wt. %, particularly from about 6.0 to about 10.0 wt. %.

In another most preferred embodiment, an inventive agent is exemplified in that it contains—relative to the total weight of the agent—(a) from about 0.1 to about 30.0 wt. %, preferably from about 1.5 to about 15.0 wt. %, more preferably from about 3.5 to about 12.0 wt. %, particularly from about 6.0 to about 10.0 wt. % formamidine sulfinic acid.

(b) Compound of Formula (I)

As a second inventive ingredient (b), the inventive agent contains at least one compound of formula (I)

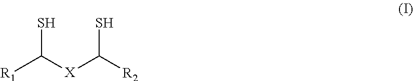

wherein
R1, R2 denote independently of one another a hydrogen atom, a carboxy group (—COOH) or the salt thereof, a C₁-C₆ alkyl group, a hydroxy C₁-C₆ alkyl group, or a C₁-C₆ alkoxy-carbonyl group,
X denotes a bivalent organic radical of formula (II) or a direct bond,

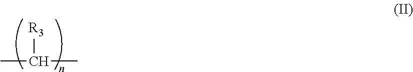

R3 denotes a hydroxy group, a hydrogen atom, a carboxy group
(—COOH) or the salt thereof, a C₁-C₆ alkoxy-carbonyl group, a
C₁-C₆-alkyl group or an amino group, and
n denotes an integer from about 1 to about 4.

Examples of the substituents R1, R2 and R3 stated in formula (I) are shown below: Examples of C₁-C₆-alkyl groups are —CH₃, —CH₂CH₃, —CH₂CH₂CH₃, —CH(CH₃)₂, —CH₂CH₂CH₂CH₃, —CH₂CH(CH₃)₂, —CH(CH₃)CH₂CH₃, —C(CH₃)₃, —(CH₂)₄CH₃, —(CH₂)₅CH₃. Even more preferred alkyl radicals are methyl and ethyl.

Examples of hydroxy-C₁-C₆-alkyl groups are —CH₂—OH, —CH₂—CH₂—OH, —CH₂—CH₂—CH₂—OH, —CH₂—CH(OH)—CH₃, —CH₂—CH₂—CH₂—CH₂—OH, the groups —CH₂—OH and —CH₂—CH₂—OH being particularly preferred.

Examples of C₁-C₆-alkoxy-carbonyl groups are the methoxycarbonyl group
(—C(O)OCH₃) and the ethoxycarbonyl group (—C(O)OCH₂CH₃).

The carboxyl group is the group —COOH. The hydrogen atom of the carboxyl group is acidic, and therefore the (protonated) carboxyl group—more particularly in a hydrous cosmetic carrier—can also be in equilibrium with its deprotonated form. Using the carboxy group in the form of its salt, such as sodium carboxylate (—COONa), potassium carboxylate (—COOK) and/or ammonium carboxylate (—COO(NH₄)) is likewise contemplated herein.

In the compounds of formula (I), radicals R1 and R2 denote independently of one another a hydrogen atom, a carboxy group (—COOH) or the salt thereof, a C₁-C₆-alkyl group, a hydroxy-C₁-C₆-alkyl group, or a C₁-C₆-alkoxy-carbonyl group.

Since radicals R1 and R2 denote, independently of one another, the aforementioned radicals, R1 and R2 can be the same or different in a compound of formula (I).

It has been shown that the compounds of formula (I), in which R1 and R2 denote independently of one another a hydrogen atom or a carboxy group, have the best decolorizing effect. Therefore, applying an agent as contemplated herein containing (b) at least one compound of formula (I), wherein R1 and R2 denote, independently of one another, a hydrogen atom or a carboxy group (—COOH) (and/or the salt thereof) is particularly preferred.

In an even more preferred embodiment, an inventive is exemplified in that it (a) contains at least one compound of formula (I), wherein

R1, R2 denote, independently from one another, a hydrogen atom, a carboxy group (—COOH) or the salt thereof.

In another most preferred embodiment, radicals R1 and R2 denote identical substituents.

In an even more preferred embodiment, an agent as contemplated herein is exemplified in that it (a) contains at least one compound of formula (I), wherein

R1, R2 both denote a hydrogen atom.

In the compounds of formula (I), X denotes either a bivalent organic radical of formula (II) or a direct bond

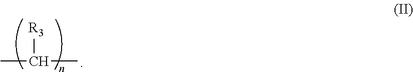

The grouping of formula (II) is a bivalent radical, i.e. the radical placed between parentheses is bound—via the bond drawn on the left and on the right respectively—to the carbon atoms of formula (I) carrying the thiol group.

The radical n can denote an integer from about 1 to about 4 and indicates the number of units —CH(R₃)— comprising the structural unit (II).

Where n denotes 1 (and R₃ a hydrogen atom), the grouping of Formula (II) is a methylene group.

Where n denotes 2 (and R₃ a hydrogen atom), the grouping of Formula (II) is an ethylene group.

Thus, where X denotes a grouping of formula (II), the compounds as contemplated herein are compounds of formula (Ia)

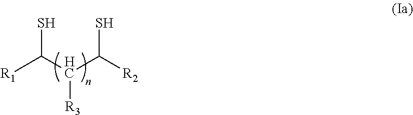

Radical R3 denotes a hydroxy group, a hydrogen atom, a carboxy group (—COOH) or the salt thereof, a C₁-C₆ alkoxy-carbonyl group, a C₁-C₆-alkyl group or an amino group.

In decolorizing tests, the compounds of formula (I), wherein X denotes a bivalent organic radical of formula (II) and the radical R3 denotes a hydroxy group or a hydrogen atom, have proven particularly potent. Explicitly most preferably, R3 denotes a hydroxy group.

In another more preferred embodiment, an agent as contemplated herein is exemplified in that it (b) contains at least one compound of formula (I), wherein

X denotes a bivalent organic radical of formula (II),

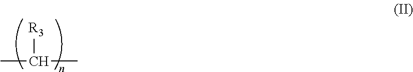

and
R3 denotes a hydroxy group or a hydrogen atom.

In decolorizing tests, the compounds of formula (I), wherein X denotes a bivalent organic radical of formula (II) and n denotes the number 2 or 3, have proven particularly effective. Explicitly most preferably, n denotes the number 2.

In another more preferred embodiment, an agent as contemplated herein is exemplified in that it (b) contains at least one compound of formula (I), wherein

X denotes a bivalent organic radical of formula (II),

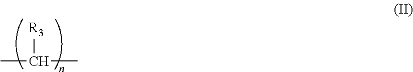

and
n denotes the number 2 or 3, more preferably the number 2.

Moreover, the compounds of formula (I), wherein the radical X denotes a direct bond, have proven highly suitable. Where X denotes a direct bond, the compounds as contemplated herein are compounds of formula (Ib)

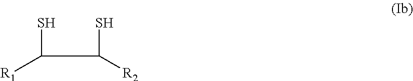

Certain compounds (b) of formula (I)—in combination with the formamidine sulfinic acid (salts) (a) are able to achieve a particularly good decolorization of artificially-dyed hair. Therefore, the use of one or more compounds from the following group is most preferred

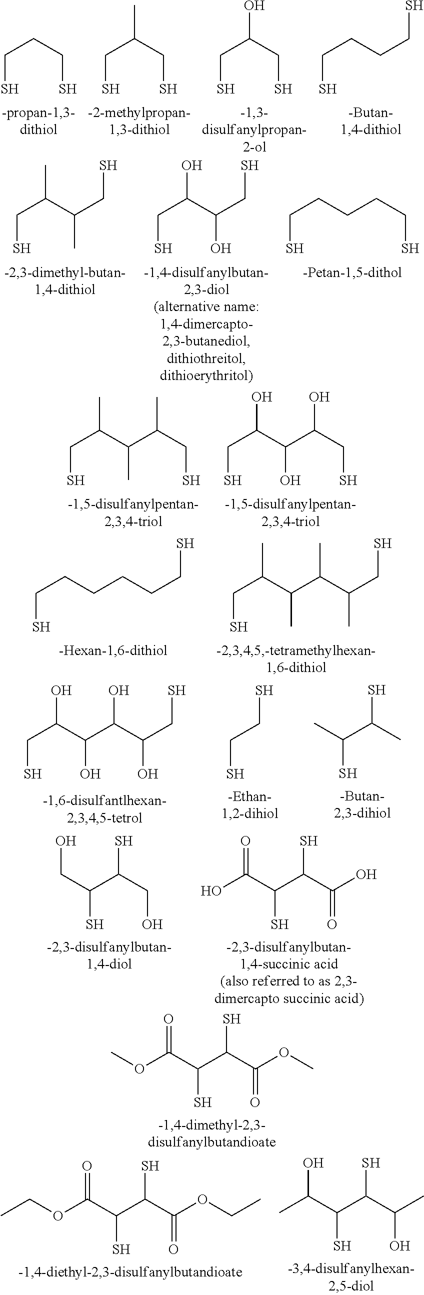

In a particularly preferred embodiment, an agent as contemplated herein is exemplified in that it (b) contains at least one compound of formula (I), which is selected from the group of propan-1,3-dithiol, 2-methylpropan-1,3-dithiol, 1,3-disulfanylpropan-2-ol, butan-1,4-dithiol, 2,3-dimethyl-butan-1,4-dithiol, 1,4-disulfanylbutan-2,3-diol (1,4-dimercapto-2,3-butanediol, dithiothreitol, dithioerythritol), pentan-1,5-dithol, 2,3,4-trimethyl-pentan-1,5-dithiol, 1,5-disulfanylpentan-2,3,4-triol, hexan-1,6-dithiol, 2,3,4,5-tetramethylhexan-1,6-dithiol, 1,6-disulfanylhexan-2,3,4,5-tetrol, ethan-1,2-dihiol, butan-2,3-dithiol, 2,3-disulfanylbutan-1,4-diol, 2,3-disulfanyl-1,4-butane dioic acid, 1,4-dimethyl-2,3-disulfanylbutandioate, 1,4-diethyl-2,3-disulfanylbutandioate and/or 3,4-disulfanylhexan-2,5-diol, is applied to the dyed keratinous fibers.

In a particularly preferred embodiment, an agent as contemplated herein is exemplified in that it (b) contains at least one compound of formula (I), which is selected from the group of

1,3-disulfanylpropan-2-ol

1,4-disulfanylbutan-2,3-diol (1,4-dimercapto-2,3-butanediol, dithiothreitol, tol)

1,5-disulfanylpentan-2,3,4-triol

2,3-disulfanylbutan-1,4-diol

2,3-disulfanyl-1,4-succinic acid (dimercapto succinic acid)

1,4-dimethyl-2,3-disulfanylbutandioate and/or

1,4-diethyl-2,3-disulfanylbutandioate

Most of all and explicitly most preferred is 1,4-disulfanylbutan-2,3-diol (1,4-dimercapto-2,3-butanediol, dithiothreitol, dithioerythritol).

The compounds of Formula (I) as contemplated herein can—depending on the substitution method thereof—contain one or more carbon atoms, which carry 4 different substituents. If this is the case, a compound of formula (I) can occur in the form of various stereoisomers. Essentially, stereoisomers have the same structure

(i.e. constitution)—and hence the same molecular formula—but differ in the spatial arrangement (configuration) of the atoms.

For example, dithiothreitol (1,4-disulfanylbutan-2,3-diol) can occur in the form of two stereoisomers, in the (2S,3S)-form and in the (2R,3R)-form. Moreover, 1,4-disulfanylbutan-2,3-diol also occurs in a form known as dithioerythritol or dithioerythrite, this being the meso-form.

This present disclosure explicitly comprises all stereoisomers.

The compounds (b) of formula (I) are used in the agent as contemplated herein in specific quantity ranges. A decolorizing effect can be observed from small application quantities. To obtain an adequate and strong decolorizing effect, however, it is advantageous for the decolorizing agent to contain one or more dithio compounds of formula (I) in a total quantity from about 0.1 to about 30.0 wt. %, preferably from about 0.2 to about 20.0 wt. %, more preferably from about 0.3 to about 10.0 wt. % and most preferably from about 0.5 to about 6.0 wt. %. The calculation basis for the quantity values in wt. % is the total weight of all reducing agents of formula (I) contained in the agent, said reducing agents being used relative to the total weight of the agent.

In a further most preferred embodiment, an agent as contemplated herein is exemplified in that it contains—relative to the total weight of the agent—(b) one or more compounds of formula (I) in a total amount of from about 0.1 to about 30.0 wt. %, preferably from about 0.2 to about 20.0 wt. %, more preferably from about 0.3 to about 10.0 wt. %, particularly from about 0.5 to about 6.0 wt. %.

Ratio of (a) to (b)

Using the combination of formamidine sulfinic acid (salts) (a) and disulfide compounds (b) of formula (I), the speed of the reductive color removal can be increased and the application duration can be optimized.

If both ingredients (a) and (b) are used together in the agent, a significant and satisfactory decolorization can already be observed after a short application time (from about 10-about 30 minutes). Without being limited to this theory, it is assumed that the two reducing agents (a) and (b) interact synergistically and that the addition of dithiols (b) enhances the formamidine sulfinic acid (a).

In this connection, it was found that ingredients (a) and (b) interact best when they are used in a specific ratio to each other.

It has been found to be particularly advantageous in this connection if the weight ratio of (a) the total amount of all formamidine sulfinic acid (salts) to (b) the total amount of all compounds of formula (I) contained in the agent, i.e. the weight ratio (a)/(b), has a value of from about 1 to about 10, preferably from about 2 to about 8, particularly from about 3 to about 6.

In another particularly preferred embodiment, an agent as contemplated herein is exemplified in that the weight ratio of (a) the total amount of all formamidine sulfinic acid (salts) to (b) the total amount of all compounds of formula (I) contained in the agent, i.e. the weight ratio (a)/(b), has a value of from about 1 to about 10, preferably from about 2 to about 8, particularly from about 3 to about 6.

Example: A ready-to-use decolorizing agent (100 g) contains

(a) 8.0 g formamidine sulfinic acid and
(b) 2.0 g 1,4-disulfanylbutan-2,3-diol (1,4-dimercapto-2,3-butanediol)
The weight ratio (a)/(b) is (8.0 g/2.0 g)=4.0.

Surfactants

The agents as contemplated herein can also contain at least one surfactant from the anionic, amphoteric, zwitterionic, non-ionic and/or cationic surfactants.

In another most preferred embodiment, an agent as contemplated herein is exemplified in that an agent containing at least one surfactant from the group of anionic, amphoteric, zwitterionic, non-ionic and/or cationic surfactants is applied to the dyed keratinous fibers.

Surfactants are amphiphilic (bifunctional) compounds that include at least one hydrophobe and at least one hydrophilic molecular part. The hydrophobic radical is preferably a hydrocarbon chain with 8-24 carbon atoms, which can be saturated or unsaturated, linear or branched. This C₈-C₂₄ alkyl chain is most preferably linear.

Accordingly, preferred anionic surfactants are exemplified by the presence of a water-solubilizing anionic group, such as a carboxylate or phosphate group and a lipophilic alkyl group having approximately 8 to 30 carbon atoms. Furthermore, the molecule can contain glycol or polyglycol ether groups, ester, ether and amide groups, as well as hydroxyl groups.

Typical examples of anionic surfactants are mono and dialkyl sulfosuccinates, mono and amide soaps, ether carboxylic acids and the salts thereof, fatty acid ethionates, fatty acid sarcosinates, fatty acid taurides, acyllactylates, acyltartrates, acylglutamates, acylaspartates, protein fatty acid condensates (more particularly wheat-based plant products) and alkyl(ether)phosphates. Insofar as anionic surfactants contain polyglycolether chains, they can have a conventional, preferably however a constricted, homologous distribution.

Examples of anionic surfactants as contemplated herein are, each in the form of sodium, potassium and ammonium salts, as well as mono-, di- and trialkanol ammonium salts having from about 2 to about 4 carbon atoms in the alkanol group,

Linear and branched fatty acids having from about 8 to about 30 carbon atoms (soaps),

Ether carboxylic acids having the formula R—O—(CH₂—CH₂O)ₓ—CH₂—COOH, wherein R is a linear alkyl group having from about 8 to about 30 carbon atoms and x=0 or from about 1 to about 16,

Acyl sarcosides having from about 8 to about 24 carbon atoms in the acyl group,

Acyl tauride having from about 8 to about 24 carbon atoms in the acyl group,

Acylisethionates with from about 8 to about 24 carbon atoms in the acyl group, which are accessible through the esterification of fatty acids with the sodium salt of the 2-hydroxyethane sulfonic acid (isethionic acid), If fatty acids having from about 8 to about 24 carbon atoms, e.g. lauric, myristic, palimitic or stearic acid or also technical fatty acid fractions, e.g. the C₁₂-C₁₈ fatty acid fractions obtainable from coconut fatty acid are used for said esterification, the C₁₂-C₁₈ acylisethionates preferably suitable as contemplated herein are obtained,

Esters of tartaric acid and citric acid having alcohols, which are the addition products of about 2-15 molecules of ethyl oxide and/or propylene oxide on fatty alcohols having from about 8 to about 22 carbon atoms,

Alkyl- and/or alkenyletherphosphates of formula

R¹(OCH₂CH₂)ₙ—O—(PO—OX)—OR²,

in which R¹ preferably denotes an aliphatic hydrocarbon radical with from about 8 to about 30 carbon atoms, R² denotes hydrogen, a radical (CH₂CH₂O)ₙR² o X, n denotes integers from about 1 to about 10 and X denotes hydrogen, an alkali- or earth alkali metal or NR³R⁴R⁵R⁶, with R³ to R⁶ denote independently hydrogen or a C₁-C₄ hydrocarbon residue,

Sulfated fatty acid alkylene glycol esters of the formula RCO(AlkO)ₙSO₃M in which RCO denotes a linear or branched, aliphatic, saturated and/or unsaturated acyl radical with from about 6 to about 22 carbon atoms, Alk denotes CH₂CH₂, CHCH₃CH₂ and/or CH₂CHCH₃, n denotes integers from about 0.5 to about 5 and M denotes a metal, such as an alkali metal, more particularly sodium, potassium, lithium, earth alkali metal, more particularly magnesium, calcium, zinc, or ammonium ion, such as ⁺NR³R⁴R⁵R⁶, with R³ to R⁶ denoting independently hydrogen or a C1-C4 hydrocarbon radical,

Monoglyceride sulfates and monoglyceride ethersulfates of formula

R⁸OC—(OCH₂CH₂)ₓ—OCH₂—[CHO(CH₂CH₂O)_{y}H]—CH₂O(CH₂CH₂O)_{z}—SO₃X,

in which R⁸CO denotes a linear or branched acyl radical with from about 6 to about 22 carbon atoms, x, y and z in total denotes 0 or integers from about 1 to about 30, more preferably from about 2 to about 10, and X denotes an alkali- or earth alkali metal, Typical examples of monoglyceride (ether) sulfates suitable for the purposes of the present disclosure are the reaction products of lauric acid monoglyceride, coconut fatty acid monoglyceride, palmitic acid monoglyceride, stearic acid monoglyceride, oleic acid monoglyceride and tallow fatty acid monoglyceride, and also the ethylene oxide adducts thereof sulfur trioxide or chlorosulfonic acid in the form of their sodium salts. Preferably, monoglyceride sulfates are used, wherein R⁸CO denotes a linear acyl radical having from about 8 to about 18 carbon atoms.

Amidethercarbonic acids, Formula R¹—CO—NR²—CH₂CH₂—O—(CH₂CH₂O)ₙCH₂COOM, with R¹ as straight-chained or branched alkyl- or alkenyl radical with a number of carbon atoms in the chain from about 2 to about 30, n denotes an integer from about 1 to about 20 and R² denotes hydrogen, a methyl-, ethyl-, propyl-, isopropyl-, n-butyl-, t-butyl- or iso-butyl radical and M stands for hydrogen or a metal such as an alkali metal, more particularly sodium, potassium, lithium, earth alkali metal, more particularly magnesium, calcium, zinc, or an ammonium ion, such as ⁺NR³R⁴R⁵R⁶, with R³ to R⁶ denoting independently hydrogen or a C1-C4 hydrogen radical. Such products can be obtained from the company Chem-Y, for example, under the product designation Akypo®, and

Acylglutamates of formula XOOC—CH2CH2CH(C(NH)OR)—COOX, in which R21CO denotes a linear or branched acyl radical with from about 6 to about 22 carbon atoms and 0 and/or 1, 2 or 3 double bonds and X for hydrogen, an alkali and/or earth alkali metal, ammonium, alkylammonium, alkanolammonium or glucammonium.

The agent as contemplated herein can contain one or more amphoteric and/or zwitterionic surfactants. In the case of the zwitterionic surfactants, the hydrophilic molecule comprises a zwitterionic structural unit, i.e. a structural unit comprising both a cationically-charged and also an anionically-charged molecule. As contemplated herein, particularly suitable zwitterionic surfactants (b) are exemplified in that they have a cationically-charged molecule in the form of a quaternary ammonium group and their anionic molecule exists in the form of a grouping or —COO⁻.

An ammonium group is quaternary when a type (RₐR_{b}R_{c}R_{d}N)⁺ grouping exists, i.e. when all four H-atoms of the NH₄ ion from which the quaternary ammonium group is derived, is replaced by organic radicals R (and/or Rₐ to R_{d}).

Particularly suitable zwitterionic surfactants include betaines, N-alkyl-N,N-dimethylammonium-glycinates, N-acyl-aminopropyl-N,N-dimethylammoniumglycinates, and 2-alkyl-3-carboxymethyl-3-hydroxyethyl-imidazolines. Suitable agents as contemplated herein are also exemplified in that the agent additionally contains at least one amphoteric surfactant. Preferred amphoteric surfactants are n-alkylglycines, n-alkylpropionic acids, n-alkylaminobutyric acids, n-alkyliminodipropionic acids, n-hydroxyethyl-n-alkylamidopropylglycines, n-alkyltaurines, n-alkyl sarcosines, 2-alkylaminopropionic acids and alkylaminoacetic acids. Particularly preferred amphoteric surfactants are N-cocosalkylaminopropionate, cocosacylaminoethylaminopropionate and C₁₂-C₁₈ acylsarcosin.

It has also proved advantageous for the agent to contain other, non-ionogenic surfactants. Preferred non-ionic surfactants are alkyl polyglycosides as well as alkylene oxide binding agents to fatty alcohols, fatty acids and fatty acid glycerides with from about 2 to about 50 moles of ethylene oxide per mole of fatty alcohol or fatty acid. Preparations with outstanding properties are also obtained if they contain fatty acid esters of ethoxylated glycerol as nonionic surfactants. The most preferred embodiment is the agent containing as a non-ionic surfactant an ethoxylated castor oil with from about 2 to about 50 mol ethylene oxide per mol of fatty acid or an ethoxylated, hydrated castor oil with from about 2 to about 50 mol ethylene oxide per mol of fatty acid. The use of PEG-40 Castor Oil is most preferred in this context.

The agents as contemplated herein can contain one or more cationic surfactants. Cationic surfactants are surfactants, i.e. surfactant compounds, each having one or more positive charges. Cationic surfactants contain exclusively positive charges. Usually, said surfactants are constructed from a hydrophobic part and a hydrophilic head group, wherein the hydrophobic part normally includes a hydrocarbon structure (e.g. including one or two linear or branched alkyl chains), and the positive charge(s) are localized in the hydrophilic head group. Cationic surfactants adsorb on boundary surfaces and aggregate in hydrous solutions above the critical micelle formation concentration to form positively charged micelles.

Examples of cation surfactants are

quaternary ammonium bonds, which can carry, as hydrophobic radicals, one or two alkyl chains with a chain length of from about 8 to about 28 carbon atoms

quaternary phosphonium salts, substituted with one or more alkyl chains with a chain length of from about 8 to about 28 carbon atoms or

Moreover, the cationic charging can also occur in the form of an onium structure component of a heterocyclical ring (e.g. of an imidazolium ring or a pyridinium ring).
In addition to the functional unit carried by the cationic charge, the cationic surfactant can also contain other uncharged functional groups, which is the case with esterquats, for example.

As contemplated herein, preferred cationic surfactants are of the type of quaternary ammonium compounds, esterquats and amidoamines. Preferred quaternary ammonium compounds are ammonium compounds and ammonium halides, more particularly chlorides and bromides, such as alkyltrimethylammoniumchlorides, dialkyldimethylammoniumchlorides and trialkylmethylammoniumchlorides, e.g. cetyltrimethylammoniumchloride, stearyltrimethylammoniumchloride, distearyldimethylammoniumchloride, lauryldimethylammoniumchloride, lauryldimethylbenzylammoniumchloride and tricetylmethylammoniumchloride, as well as the imidazolium compounds known under the INCI trade names of Quaternium-27 and Quaternium-83. The long alkyl chains of the aforementioned surfactants preferably have from about 10 to about 18 carbon atoms.

Esterquats are known substances containing both at least one ester function and at least quaternary ammonium group as the structural element. Preferred esterquats are quaternated ester salts of fatty acids with triethanolamine, quaternated ester salts of fatty acids with diethanolalkyl amines and quaternated ester salts of fatty acids with 1,2-dihydroxypropyldialkylamines. The alkylamidoamines are usually produced through the amidation of natural or synthetic fatty acids and fatty acid molecules with dialkylaminoamines. A most preferred compound from this substance group as contemplated herein is the stearamidopropyldimethylamine commercially available under the trade name of Tegoamid® S 18. As contemplated herein, the quaternized protein hydrolysates can also be used.

The anionic, amphoteric, zwitterionic, non-ionic and cationic surfactants may be contained in a total quantity of from about 0.1 to about 15.0 wt. %, preferably from about 0.25 to about 12.0 wt. %, more preferably from about 1.25 to about 10.0 wt. % and most preferably from about 1.50 to about 9.0 wt. %—calculated based on the total weight of the agent in each case.

Polyols

The use of polyols can further support the decolorizing effect. For this reason, the decolorizing agent as contemplated herein preferably contains one or more polyols.

A polyol is a compound having at least two aliphatic (i.e. non-phenolic) OH groups.

Examples of suitable polyols as contemplated herein are, in particular, ethylene glycol, 1,2-propylene glycol, 1,3-propanediol, 1,2-butanediol, 1,3-butanediol, 1,2-pentanediol, 1,3-pentanediol, 1,4-pentanediol, 1,5-pentanediol, 1,2-hexanediol, 1,3-hexanediol, 1,4-hexanediol, 1,5-hexanediol and 1,6-hexanediol. Polyethylene glycol and polypropylene glycol, however, are also suitable.

In another embodiment, a decolorizing agent as contemplated herein is therefore exemplified in that it additionally contains one or more polyols from the group of ethylenegylcol (1,2-ethanediol), 1,2-propanediol, 1,3-propanediol, 1,2-butanediol, 1,3-butanediol, 1,4-butanediol, 1,2-pentanetiol, 1,3-pentanetiol, 1,4-pentanetiol, 1,5-pentanetiol, 1,2-hexanediol, 1,3-hexanediol, 1,4-hexanetiol, 1,5-hexanediol, 1,6-hexanediol, polyethylene glycol, polypropylene glycol.

The polyols are preferably contained in the agents as contemplated herein in a total quantity of from about 0.5 to about 15.0 wt. %, more preferably from about 2.5 to about 13.5 wt. %, even more preferably from about 3.5 to about 11.5 wt. %, and most preferably from about 4.5 to about 9.5 wt. %, relative to the total weight of the cosmetic agent.

Decolorization of Dyed Keratinous Fibers or Hair

The agent as contemplated herein is used to decolorize previously dyed human keratinous fibers. The dyed keratinous fibers are fibers which have been colored beforehand by means of conventional oxidative dyes and/or partially oxidative dyes known to a person skilled in the art.

The decolorizing agents are suitable for removing colors produced on the keratinous fibers by means of oxidizing dyes based on developer and coupler components. If the following compounds were used as developers, the colors thus produced can easily be removed effectively and almost without subsequent post-darkening by means of the decolorizing agent: p-phenylendiamine, p-toluylendiamine, N,N-bis-(β-hydroxyethyl)-p-phenylendiamine, 4-N,N-bis-(β-hydroxyethyl)-amino-2-methylaniline, 2-(β-hydroxyethyl)-p-phenylendiamine, 2-(α,β-dihydroxyethyl)-p-phenylendiamine, 2-hydroxymethyl-p-phenylendiamine, bis-(2-hydroxy-5-aminophenyl)-methane, p-aminophenol, 4-amino-3-methylphenol, 2,4,5,6-tetraaminopyrimidine, 4-hydroxy-2,5,6-triaminopyrimidine, 2-hydroxy-4,5,6-triaminopyrimidine and/or 4,5-diamino-1-(β-hydroxyethyl)-pyrazole.

If the following compounds were used as couplers, the colors produced thereby can likewise be removed with very good decolorization results: m-phenylendiamine derivatives, naphthols, resorcin and resorcin derivatives, pyrazolone and m-aminophenol derivatives. Suitable coupler substances are, in particular, 1-naphthol, 1,5-, 2,7- and 1,7-dihydroxynaphthalene, 5-amino-2-methylphenol, m-aminophenol, resorcinol, resorcin monomethyl ether, m-phenylenediamine, 1-phenyl-3-methyl-pyrazolone-5, 2,4-dichloro-3-aminophenol, 1,3-bis-(2′, 4′-diaminophenoxy)-propane, 2-chloro-resorcin, 4-chloro-resorcin, 2-chloro-6-methyl-3-aminophenol, 2-amino-3-hydroxypyridine, 2-methylresorcinol, 5-methylresorcinol and 2-methyl-4-chloro-5-aminophenol, 1-naphthol, 1,5-dihydroxynaphthalene, 2,7-dihydroxynaphthalene, 1,7-dihydroxynaphthalene, 3-aminophenol, 5-amino-2-methylphenol, 2-amino-3-hydroxypyridine, resorcinol, 4-chlororesorcinol, 2-chloro-6-methyl-3-aminophenol, 2-methylresorcinol, 5-methylresorcinol, 2,5-dimethylresorcin and 2,6-dihydroxy-3,4-dimethylpyridine.

Certain dyes and/or dye combinations can be removed particularly well and particularly completely using ingredients (a) and (b).

It has been found, for example, that the natural tints otherwise known for their durability and resistance can be decolorized particularly well if an agent with (a) formamidine sulfinic acid and (b) at least one dithiol of formula (I) are used.

The decolorization was particularly effective on hair that was dyed with one of the following combinations of developers/couplers:

p-toluylendiamine/resorcin, p-toluylendiamine/2-methylresorcin, p-toluylendiamine/4-resorcin, p-toluylendiamine/m-aminophenol, N,N-bis-(β-hydroxyethyl)-p-phenylendiamine/resorcin, N,N-bis-(β-hydroxyethyl)-p-phenylendiamine/2-methylresorcin, N,N-bis-(β-hydroxyethyl)-p-phenylendiamine/4-chlorresorcin, N,N-bis-(β-hydroxyethyl)-p-phenylendiamine/m-aminophenol 2-(β-hydroxyethyl)-p-phenylendiamine/resorcin, 2-(β-hydroxyethyl)-p-phenylendiamine/2-methylresorcin, 2-(β-hydroxyethyl)-p-phenylendiamine/4-chlorresorcin and/or 2-(β-hydroxyethyl)-p-phenylendiamine/m-aminophenol.

In another preferred embodiment, an agent as contemplated herein is exemplified in that the keratinous fibers were previously dyed by means of an agent containing at least one of the following combinations: p-toluylendiamine/resorcin, p-toluylendiamine/2-methylresorcin, p-toluylendiamine/4-chlorresorcin, p-toluylendiamine/m-aminophenol, N,N-bis-(β-hydroxyethyl)-p-phenylendiamine/resorcin, N,N-bis-(β-hydroxyethyl)-p-phenylendiamine/2-methylresorcin, N,N-bis-(β-hydroxyethyl)-p-phenylendiamine/4-chlorresorcin, N,N-bis-(β-hydroxyethyl)-p-phenylendiamine/m-aminophenol,2-(β-hydroxyethyl)-p-phenylendiamine/resorcin, 2-(β-hydroxyethyl)-p-phenylendiamine/2-methylresorcin, 2-(β-hydroxyethyl)-p-phenylendiamine/4-chlorresorcin and/or 2-(β-hydroxyethyl)-p-phenylendiamine/m-aminophenol.

The decolorizing agents as contemplated herein are designed to remove said colors and therefore themselves preferably contain no dyes, more particularly no oxidative dye precursors of the developer type and/or coupler type, as well as partially-oxidizing dyes.

In another most preferred embodiment, an agent as contemplated herein is exemplified in that an agent, wherein the total amount of all partially-oxidizing dyes and oxidation dye precursors has a maximum value of 0.2 wt. %, preferably 0.1 wt. %, more preferably 0.05 wt. %, particularly 0.01 wt. %—relative to the total weight of the agent.

pH Value

For the application, the decolorizing agent as contemplated herein is preferably set to the pH value at which is develops its greatest effect. Formamidine sulfinic acid (a) achieves its optimal effect in the alkaline range. For storage purposes, however, it may be preferable to select the pH value at which the ingredients (a) and (b) are most stable. Since the pH values suitable for storage and application usually differ, changing the pH value of the decolorizing agent shortly before application can be particularly advantageous.

During the work that led to this present disclosure, it emerged that the pH value for achieving a particularly strong decolorizing effect is a key determinant. It has emerged that the ready-to-use decolorizing agents most preferably has a pH value in the range from about 7.5 to about 12.5.

A decolorizing effect was also achieved by means of acidically or neutrally set agents. However, it was observed that the strongest decolorizing effect is achieved by treating the hair with an agent set to a pH value above 7.5, preferably above 8.0, and more preferably above 8.5. However, setting pH values above 11.0 is avoided in order to prevent excessive hair damage and also increased skin irritation.

In another most preferred embodiment, an agent as contemplated herein is exemplified in that it contains water and has a pH value in the range from about 7.5 to about 12.5, preferably from about 8.0 to about 10.5, particularly from about 8.5 to about 10.0.

The pH value can be measured by means of a gas electrode, for example, which is usually commercially available in the form of a combination electrode. Before the pH value is measured, the gas electrodes are usually calibrated with calibration solutions of a known pH value. The pH values as defined by the present disclosure are pH values that were measured at a temperature of 22° C.

The desired pH value can be set by means of various alkalizing agents. Suitable alkalizing agents as contemplated herein are selected from the group formed from ammonia, alkanolamines, alkali metal hydroxides, alkali metal metasilicates, alkalimetal phosphates and alkali metal hydrogen phosphates. Preferred inorganic alkalizing agents are sodium hydroxide, sodium hydroxide, sodium silicate and sodium metasilicate. Organic alkalizing agents usable as contemplated herein are preferably selected from monoethanolamine, 2-amino-2-methylpropanol and triethanolamine. The basic amino acids that can likewise be used as alkalizing agents are preferably can be selected from the group formed of arginine, lysine, ornithine, and histidine.

Although the hair treatment agents as contemplated herein are preferably set to pH values in the alkali range, it may be necessary to also use small quantities of acidification agents in order to finely adjust the pH value. Acidification agents suitable as contemplated herein include citric acid, lactic acid, acetic acid and diluted mineral acids (such as hydrochloric acid, sulfuric acid, phosphoric acid).

Multi-Component Package Unit (Kit-Of-Parts)

The ready-to-use decolorizing agent is preferably produced by mixing two or multiple—previously separately packaged—agents. It is particularly comfortable for the user if these different agents are provided in the form of a multi-component package unit (kit-of-parts, also referred to as a kit).

In order to provide a powerful agent that is stable during storage, it has been found to be particularly advantageous if the ready-to-use decolorizing agent is produced shortly before the planned use and the formamidine sulfinic acid (a), dithiols (b) and alkalizing agents (a) and (b) used for activation of the reducing agent are stored separately.

In a particularly preferred embodiment, the ready-to-use decolorizing agent is produced shortly before use by mixing formamidine sulfinic acid (salt) (a), dithiol (b) and alkalizing agent (c). In the connection, different types of packaging have been found to be particularly suitable.

For example, the ready-to-use decolorizing agent can be produced by mixing two components. In the context of an embodiment, it is preferable to incorporate the formamidine sulfinic acid (a) and the dithiol(s) (b) together into an agent which is store in a first container. In order to adjust a particularly advantageous pH value range for use, this first agent can be mixed with a second agent adjusted to alkaline.

A further subject of the present disclosure, therefore, is a multi-component package unit (kit-of-parts) for reductive decolorizing of dyed keratinous fibers, particularly human hair, which are packaged separately from each other, comprising

a first container (1) containing a cosmetic agent (1) and

a second container (2) containing a cosmetic agent (2), wherein

agent (1) in container (1) contains

(a) Formamidine sulfinic acid and/or salts thereof and
(b) At least one compound of formula (I),

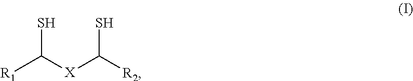

where radicals R1, R2 and X were disclosed in the description of the subject of the present disclosure
and

agent (2) in container (2)

(c) Contains one or multiple alkalizing agents.

As contemplated herein, it is also feasible to incorporate only the formamidine sulfinic acid (salts) (a) into a first agent and to dispense it into a first container (1). Then this first agent can be mixed with a second agent in a second container shortly before use. This second agent contains both the dithiol(s) (b) of formula (I) and the alkalizing agent or agents (c).

A further subject of the present disclosure, therefore, is a multi-component package unit (kit-of-parts) for reductive decolorizing of dyed keratinous fibers, particularly human hair, which are packaged separately from each other, comprising

a first container (1) containing a cosmetic agent (1) and

a second container (2) containing a cosmetic agent (2), wherein

agent (1) in container (1) contains

(a) Formamidine sulfinic acid and/or salts thereof and

agent (2) in container (2)

(b) At least one compound of the formula (I),

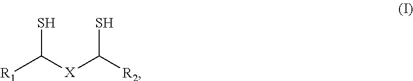

where radicals R1, R2 and X were disclosed in the description of the subject of the present disclosure,
and
(c) Contains one or multiple alkalizing agents.

The ready-to-use decolorizing agent is now produced by mixing agents (1) and (2). In principle, agents (1) and (2) can be mixed in various mixing ratios, such as (1)/(2) from about 20:1 to about 1:20. Agents (1) and (2) are preferably mixed with one another in a mixing ratio of from about 1:10 to about 10:1, more preferably from about 1:2 to about 2:1. The mixing ratios indicate the ratio of the weight quantities of the preparations (1) and (2) relative to one another.

To produce the ready-to-use mixture, the agent (1) can be transferred from container (1) completely to container (2)—which already contains the agent (2). In this case, the size of container (2) is selected such that container (2) can receive the total quantity of the preparations (1) and (2) and also allows the agents (1) and (2) to be mixed by shaking or stirring, for example.

Likewise, the mixture can also be produce by completely transferring agent (2) from container (2) to container (1)—which already contains the agent (1). In this case, the size of container (1) should be selected such that container (1) can receive the total quantity of the preparations (1) and (2) and also allows the two agents (1) and (2) to be mixed by shaking or stirring, for example.

The ready-to-use agent can also be produced by mixing three separately packaged agents. Within this embodiment, the inventive multi-component package unit comprises a first agent (1) in a first container (1), a second agent (2) in a second container (2) and a third agent (3) in a third container (3). Agent (1) contains the formamidine sulfinic acid (salts) (a), agent (2) contains the dithiol(s) (b) of formula (I) and agent (3) contains one or multiple alkalizing agents (c).

A further subject of the present disclosure, is a multi-component package unit (kit-of-parts) for reductive decolorizing of keratinous fibers, particularly human hair, comprising, separately packaged:

a first container (1) containing a cosmetic agent (1) and

a second container (2) containing a cosmetic agent (2) and

a third container (3) containing a cosmetic agent (3), wherein

agent (1) in container (1) contains

(a) Formamidine sulfinic acid and/or salts thereof and

agent (2) in container (2)

(b) At least one compound of the formula (I),

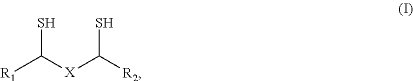

where radicals R1, R2 and X were disclosed in the description of the subject of the present disclosure, and

agent (3) in container (3)

(c) Contains one or multiple alkalizing agents.

The ready-to-use decolorizing agent can be produced by mixing the three agents (1) and (2) and (3). For example, agents (1) and (2) can be mixed first, then this mixture is combined with agent (3). In principle, agents (1), (2) and (3) can also be used here mixed in various mixing ratios, such as (1)/(2)/(3) from about 1:1:10 to about 1:10:1 to about 10:1:1. Agents (1) and (2) are preferably mixed with one another in a mixing ratio of approximately 1:1:1, 2:1:1, 1:2:1 or 1:1:2. The mixing ratios indicate the ratio of the total quantities of the preparations (1), (2) and (3) relative to one another.

Particularly good results could be obtained when at least one alkalizing agent (c) was selected from the group of ammonia, 2-aminoethan-1-ol, 2-amino-2-methylpropan-1-ol, arginine, lysine, ornithine, histidine, potassium hydroxide, sodium hydroxide, magnesium hydroxide and/or calcium hydroxide. In a further particularly preferred embodiment, an inventive multicomponent package unit is exemplified in that alkalizing agent (c) is selected from the group of ammonia, 2-aminoethan-1-ol, 2-amino-2-methylpropan-1-ol, arginine, lysine, ornithine, histidine, potassium hydroxide, sodium hydroxide, magnesium hydroxide and/or calcium hydroxide.

Agents (1) and (2) (and (3), if applicable) are packaged separately from one another and can be provided in a suitable container for the purpose. Suitable containers include glass, or more particularly, plastic bottles, jars, tubes or other suitable containers.

With respect to other preferred embodiments of the multi-component package units as contemplated herein, the statements made regarding the agent apply mutatis mutandis.

Further ingredients in the ready-to-use decolorizing agent and/or in agents (1), (2) and/or (3)

The inventive decolorizing agent and/or agents (1), (2) and (3) of the inventive multi-component package units can also contain additional ingredients and/or active ingredients.

The agent or agents (i.e. inventive decolorizing agents or agents (1), (2) and (3)) can contain, for example, one or multiple fatty constituents. It is particularly preferred that the agents contain one or multiple fatty constituents from the group of C₁₂-C₃₀ fatty alcohols C₁₂-C₃₀ fatty acid triglycerides, C₁₂-C₃₀ fatty acid monoglycerides, C₁₂-C₃₀ fatty acid diglycerides, C₁₂-C₃₀ fatty acid esters, hydrocarbons and/or silicone oils.

To the extent required by the present disclosure, “fatty constituents” are organic compounds with a water solubility at room temperature (22° C.) and atmospheric pressure (760 mmHg) of less than about 1 wt. %, preferably less than about 0.1 wt. %.

The definition of fatty constituents explicitly includes only uncharged (i.e. non-ionic) compounds. Fatty constituents have at least one saturated or unsaturated alkyl group with at least 12 carbon atoms. The molecular weight of the fatty constituents is a maximum 5000 g/mol, preferably maximum 2500 g/mol and even more preferably a maximum of 1000 g/mol. The fatty constituents are neither polyoxyalkylated nor polyglycerylated compounds. In this connection, polyalkoxylated compounds are such compounds for which 2 alkylene oxide units were implemented in the production thereof. Analogously, polyglycerated compounds are such compounds for which two glycerin units were implemented in the production thereof.

Since only nonionic substances are considered fatty constituents within the context of the present disclosure, charged components, such as fatty acids and salts thereof do not fall under the group of fatty constituents.

Preferred fatty constituents are the constituents selected from the group of C₁₂-C₃₀ fatty alcohols C₁₂-C₃₀ fatty acid triglycerides, C₁₂-C₃₀-fatty acid monoglycerides, C₁₂-C₃₀ fatty acid diglycerides, C₁₂-C₃₀ fatty acid esters and hydrocarbons.

The C₁₂-C₃₀ fatty alcohols can be saturated, one or more unsaturated, linear or branched fatty alcohols with from about 12 to about 30 carbon atoms.

Examples of preferred linear, saturated C₁₂-C₃₀ fatty alcohols are dodecan-1-ol (dodecylalcohol, laurylalcohol), tetradecan-1-ol (retradecyl alcohol, myristyl alcohol), hexadecan-1-ol (hexadecyl alcohol, cetyl alcohol, palmityl alcohol), octadecan-1-ol (octadecyl alcohol, stearyl alcohol), arachyl alcohol (eicosan-1-ol), heneicosyl alcohol (heneicosan-1-ol) and/or behenyl alcohol (docosan-1-ol).

Preferred linear, unsaturated fatty alcohols are (9Z)-octadec-9-en-1-ol (oleyl alcohol), (9E)-octadec-9-en-1-ol (elaidyl alcohol), (9Z,12Z)-cctadeca-9,12-dien-1-ol (linoleyl alcohol), (9Z,12Z,15Z)-octadeca-9,12,15-trien-1-ol (linolenoyl alcohol), gadoleyl alcohol ((9Z)-eicos-9-en-1-ol), arachidyl alcohol ((5Z,8Z,11Z,14Z)-eicosa-5,8,11,14-tetraen-1-ol), erucyl alcohol ((13Z)-docos-13-en-1-ol) and/or brassidyl alcohol ((13E)-docosen-1-ol).

The preferred typical branched fatty alcohols are 2-octyl-dodecanol, 2-hexyl-dodecanol and/or 2-butyl-dodecanol.

To the extent required by the present disclosure, a C₁₂-C₃₀ fatty acid triglyceride is the triester of the trivalent alcohol glycerin with three equivalent fatty acids. Both identically structured and different fatty acids within a triglyceride molecule can be involved in the ester formation.

To the extent required by the present disclosure, fatty acids are saturated or unsaturated, unbranched or branched, unsubstituted or substituted C₁₂-C₃₀ carboxylic acids. Unsaturated fatty acids can be unsaturated or polyunsaturated. The C—C double bond(s) of an unsaturated fatty acid can have the cis- or trans configuration.

Fatty acid diglycerides are exemplified by their particular suitability, for which at least one of the ester groups, based on glycerine, is formed with a fatty acid, which is selected from dodecan acid (laurin acid), tetradecan acid (myristine acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enic acid], oleic acid

[(9Z)-octadec-9-enic acid], elaidic acid [(9E)-octadec-9-enic acid], erucic acid [(13Z)-docos-13-enic acid], linoleic acid [(9Z, 12Z)-octadeca-9,12-dienic acid, linoleic acid [(9Z,12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-Icosa-5,8,11,14-trienoic acid] and/or nervonic acid [(15Z)-tetracos-15-enic acid].

The fatty acid triglycerides can also be from natural sources. The fatty acid triglycerides occurring in soy bean oil, peanut oil, sunflower oil, macadamia nut oil, drumstick tree oil, apricot kernel oil, manila oil and/or possibly hardened castor oil, and the mixtures thereof are particularly suitable for use in agent (a) as contemplated herein.

A C₁₂-C₃₀ fatty acid monoglyceride is the monoester of the trivalent alcohol glycerine with an equivalent fatty acid. Either the middle hydroxy group of the glycerine or the final hydroxy group of the glycerin can be esterified with the fatty acid.

The C₁₂-C₃₀ fatty acid triglycerides are exemplified by their particular suitability, for which at least one hydroxy group of the glycerine is esterified, wherein the fatty acids are selected from dodecan acid (laurin acid), tetradecan acid (myristine acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enic acid], oleic acid [(9Z)-octadec-9-enic acid], elaidic acid [(9E)-octadec-9-enic acid], erucic acid [(13Z)-docos-13-enic acid], linoleic acid [(9Z, 12Z)-octadeca-9,12-dienic acid, linoleic acid [(9Z,12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-Icosa-5,8,11,14-trienoic acid] or nervonic acid [(15Z)-tetracos-15-enic acid].

A C₁₂-C₃₀ fatty acid diglyceride is the diester of the trivalent alcohol glycerine with two equivalent fatty acids. Either the middle or a terminal hydroxy group of glycerin can be esterified with two equivalent fatty acids or both terminal hydroxy groups of glycerin are each esterified with one fatty acid. The glycerin can be esterified with two identically structured or two different fatty acids.

Fatty acid diglycerides are exemplified by their particular suitability, for which at least one of the ester groups, based on glycerine, is formed with a fatty acid, which is selected from dodecan acid (laurin acid), tetradecan acid (myristine acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enic acid], oleic acid

[(9Z)-octadec-9-enic acid], elaidic acid [(9E)-octadec-9-enic acid], erucic acid [(13Z)-docos-13-enic acid], linoleic acid [(9Z, 12Z)-octadeca-9,12-dienic acid, linoleic acid [(9Z,12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-Icosa-5,8,11,14-trienoic acid] and/or nervonic acid [(15Z)-tetracos-15-enic acid].

In the context of the present disclosure, a C₁₂-C₃₀ fatty acid ester is understood to mean the monoester from a fatty acid and an aliphatic monovalent alcohol, where the alcohol comprises up to 6 carbon atoms. Suitable alcohols include, for example, ethanol, n-propanol, isopropanol, 1-butanol, isobutanol, tert-butanol, n-pentanol, iso-pentanol or n-hexanol. Ethanol and isopropanol are preferred alcohols.

Preferred C₁₂-C₃₀ fatty acid esters are the esters with which esterification of the alcohols and/or isopropanol are formed with one of the fatty acids selected from the group of dodecan acid (lauric acid), tetradecan acid (myristine acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enic acid], oleic acid [(9Z)-octadec-9-enic acid], elaidic acid [(9E)-octadec-9-enic acid], erucic acid [(13Z)-docos-13-enic acid], linoleic acid [(9Z, 12Z)-octadeca-9,12-dienic acid, linoleic acid [(9Z,12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-Icosa-5,8,11,14-trienoic acid] and/or nervonic acid [(15Z)-tetracos-15-enic acid]. Particular preference is given to isopropyl myristate as a fatty acid ester.

Hydrocarbons are exclusively compounds including the atoms hydrocarbon and hydrogen in compounds with from about 8 to about 250 carbon atoms, preferably 150 carbon atoms. In this context, aliphatic hydrocarbons such as mineral oils, liquid paraffin oils (e.g. paraffinum liquidum or paraffinum perliquidum), isoparaffin oils, semi-solid paraffin oils, paraffin waxes, hard paraffin (paraffinum solidum), vaseline and polydecene are preferred.

In this context, liquid paraffin oils (paraffinum liquidum and paraffinum perliquidum) have proven to be particularly suitable. The most preferable hydrocarbon is paraffinum liquidum, also referred to as white oil. Paraffinum liquidum is a mixture of cleaned, saturated, aliphatic hydrocarbons, which includes mainly of hydrogen chains with a C-chain distribution from about 25 to about 35 carbon atoms.

The fatty constituents can be the cosmetic carrier of agent (a) and also—depending on the nature and amount of the fat which is used—have a great influence on the consistency of the agent. In this context, it has been found to be particularly preferable that agent (a) has one or multiple fatty constituents in a total amount of from about 10 to about 90 wt. %, preferably from about 20 to about 60 wt. % and particularly from about 25 to about 50 wt. %, where these quantity specifications are relative to total weight of the agent (a).

Furthermore, the agents can also contain one or multiple nonionic polymers.

Polymers are macromolecules having a molecular weight of at least about 1000 g/mol, preferably at least about 2500 g/mol, more preferably at least about 5000 g/mol, which includes the same, repeating organic units. Polymers are produced by polymerization of a monomer type or by polymerization of different, structurally different monomer types. If the polymer is produced by polymerization of one monomer type, it is referred to as a homopolymer. If structurally different monomer types are used in the polymerization, they are referred to as copolymers by a person skilled in the art.

The maximum molecular weight of the polymer depends on the degree of polymerization (number of polymerized monomers) and is partly determined by the polymerization method. According to the present disclosure, the maximum molecular weight of the zwitterionic polymer (d) is preferably no more than about 10⁷ g/mol, more preferably no more than about 10⁶ g/mol and even more preferably no more than about 10⁵ g/mol.

Nonionic polymers are exemplified in that they do not have any charges.

Examples of suitable nonionic polymers are vinylpyrrolidinone/vinyl acrylate copolymers, vinylpyrrolidinone, vinylpyrrolidinone/vinyl acetate copolymers, polyethylene glycols, ethylene/propylene/styrene copolymers and/or butylene/ethylene/styrene copolymers.

Moreover, the inventive agents can contain additional active, auxiliary and additive ingredients, such as anionic, zwitterionic, amphoteric and/or cationic surfactants, cationic polymers such as quaternized cellulose ethers, polysiloxanes having quaternary groups, dimethyldiallylammonium chloride copolymers, acrylamide-dimethyldiallyl ammonium chloride copolymers, dimethylaminoethyl methacrylate-vinylpyrrolidinone copolymers quaternized with diethyl sulfate, vinyl pyrrolidinone-imidazolinium methochloride copolymers and quaternized polyvinyl alcohol; zwitterionic and amphoteric polymers; anionic polymers such as, for example, polyacrylic acids or crosslinked polyacrylic acids; structurants such as glucose, maleic acid and lactic acid, hair-conditioning compounds such as phospholipids, for example lecithin and kephalins; perfume oils, dimethyl isosorbide and cyclodextrins; fibre-structure-improving active ingredients, in particular mono-, di- and oligosaccharides such as, for example, glucose, galactose, fructose, fruit sugar and lactose; dyes for staining the agent; antidandruff active ingredients such as piroctone olamine, zinc omadins and climbazole; amino acids and oligopeptides; protein hydrolysates on an animal and/or vegetable basis, and in the form of their fatty acid condensation products or optionally anionically or cationically modified derivatives; vegetable oils; light stabilizers and UV blockers; active ingredients such as panthenol, pantothenic acid, pantolactone, allantoin, pyrrolidinonecarboxylic acids and their salts and bisabolol; polyphenols, in particular hydroxycinnamic acids, 6,7-dihydroxycoumarins, hydroxybenzoic acids, catechins, tannins, leucoanthocyanidins, anthocyanidins, flavanones, flavones and flavonols; ceramides or pseudoceramides; vitamins, provitamins and vitamin precursors; plant extracts; fats and waxes such as fatty alcohols, beeswax, montan wax and paraffins; swelling and penetration substances such as glycerol, propylene glycol monoethyl ether, carbonates, hydrogen carbonates, guanidines, ureas and primary, secondary and tertiary phosphates; opacifiers such as latex, styrene/PVP and styrene/acrylamide copolymers; pearlescent agents such as ethylene glycol mono- and distearate and PEG-3 distearate; pigments and propellants such as propan-butane mixtures, N₂O, dimethyl ether, CO₂ and air. In this context, explicit reference is made to the known monographies, e.g. Kh. Schrader, Grundlagen and Rezepturen der Kosmetika [Cosmetic principles and formulas, 2nd Edition, Hüthig Buch Verlag, Heidelberg, 1989, which reflect the corresponding knowledge of a person skilled in the art.

The aforementioned additional optional ingredients can be contained in the agent, for example, in amounts of from about 0.01 to about 30.0 wt. % relative to the total weight of the respective agent.

Method for Decolorizing Dyed Keratinous Fibers

The inventive decolorizing agent describe above and the inventive multi-component package units (kit-of-parts described above can be used in a method for reductive decolorization.

A further subject of the present disclosure is a method for reductive decolorization of keratinous fibers, particularly human hair, including the following steps in the specified sequence:

(I) Application of the ready-to-use decolorizing agent on dyed keratinous fibers,
(II) Leaving the decolorizing agent in place to take effect,
(III) Rinsing the decolorizing agent off of the keratinous fibers,
where the ready-to-use decolorizing agent applied in step (I) is an agent already disclosed in the description of the first subject of the present disclosure.

In other words, particular preference is given to a method for reductive decolorizing of dyed keratinous fibers, particularly human hair, comprising the following steps in the specified sequence:

(I) Application of the ready-to-use decolorizing agent on dyed keratinous fibers,
(II) Leaving the decolorizing agent in place to take effect,
(III) Rinsing the decolorizing agent off of the keratinous fibers,
where the ready-to-use decolorizing agent applied in step (I) is an agent containing, in a cosmetic carrier,
(a) Formamidine sulfinic acid and/or salts thereof and
(b) At least one compound of the formula (I)

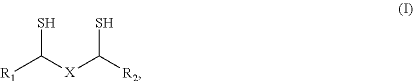

where
R1, R2 denote independently of one another a hydrogen atom, a carboxy group (—COOH) or the salt thereof, a C₁-C₆ alkyl group, a hydroxy C₁-C₆ alkyl group, or a C₁-C₆ alkoxy-carbonyl group,
X denotes a bivalent organic radical of formula (II) or a direct bond,

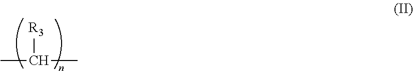

R3 denotes a hydroxy group, a hydrogen atom, a carboxy group
(—COOH) or the salt thereof, a C₁-C₆ alkoxy-carbonyl group, a
C₁-C₆-alkyl group or an amino group, and
n denotes an integer from about 1 to about 4.

Particular preference is also given to a method for reductive decolorizing of dyed keratinous fibers, particularly human hair, comprising the following steps in the specified sequence

(I) Application of the ready-to-use decolorizing agent on dyed keratinous fibers,
(ii) Leaving the decolorizing agent in place to take effect,
(iii) Rinsing the decolorizing agent off of the keratinous fibers,
where the ready-to-use decolorizing agent applied in step (I) is an agent containing, in a cosmetic carrier,
(a) Formamidine sulfinic acid and/or salts thereof and
(b) At least one compound of the formula (I)

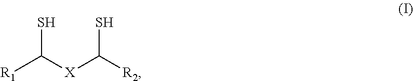

where
R1, R2 denote independently of one another a hydrogen atom, a carboxy group (—COOH) or the salt thereof, a C₁-C₆ alkyl group, a hydroxy C₁-C₆ alkyl group, or a C₁-C₆ alkoxy-carbonyl group,
X denotes a bivalent organic radical of formula (II) or a direct bond,

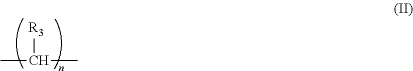

R3 denotes a hydroxy group, a hydrogen atom, a carboxy group
(—COOH) or the salt thereof, a C₁-C₆ alkoxy-carbonyl group, a
C₁-C₆-alkyl group or an amino group, and
n denotes an integer from about 1 to about 4, and
(c) One or multiple alkalizing agents.

Particular preference is also given to a method for reductive decolorizing of dyed keratinous fibers, particularly human hair, comprising the following steps in the specified sequence

(I) Application of the ready-to-use decolorizing agent on dyed keratinous fibers,
(II) Leaving the decolorizing agent in place to take effect,
(III) Rinsing the decolorizing agent off of the keratinous fibers,
where the ready-to-use decolorizing agent applied in step (I) is an agent containing, in a cosmetic carrier,
(a) Formamidine sulfinic acid and/or salts thereof and
(b) At least one compound of the formula (I)

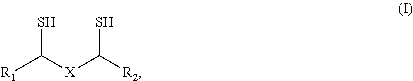

where
R1, R2 denote independently of one another a hydrogen atom, a carboxy group (—COOH) or the salt thereof, a C₁-C₆ alkyl group, a hydroxy C₁-C₆ alkyl group, or a C₁-C₆ alkoxy-carbonyl group,
X denotes a bivalent organic radical of formula (II) or a direct bond,

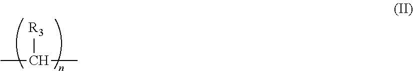

R3 denotes a hydroxy group, a hydrogen atom, a carboxy group
(—COOH) or the salt thereof, a C₁-C₆ alkoxy-carbonyl group, a
C₁-C₆-alkyl group or an amino group, and
n denotes an integer from about 1 to about 4, and
(c) One or more alkalizing agents selected from the group of ammonia, 2-aminoethan-1-ol, 2-amino-2-methylpropan-1-ol, arginine, lysine, ornithine, histidine, potassium hydroxide, sodium hydroxide, magnesium hydroxide and/or calcium hydroxide,

As described above, the use of the reducing agent combination (a) and (b) can significantly accelerate the decolorizing process. A significant advantage of the inventive method, therefore, is the shortening of the time that the ready-to-use decolorizing agent requires to take effect on the dyed keratinous fibers.

Of course, the inventive decolorizing agent can be left on the fibers to be decolorized for a longer exposure time without any detrimental effects. However, a decolorizing process of a shorter duration is particularly comfortable for the user. Therefore, it is particularly preferred that the decolorizing agent that is allowed to take effect keratinous fibers to be decolorized in step (II) of the method remains on the hair for a period of from about 10 to about 65 minutes, preferably from about 20 to about 45 minutes, particularly 20 from about to about 35 minutes.

In a further particularly preferred embodiment, an inventive method is exemplified by

(II) Leaving the decolorizing agent in place to take effect for a period of from about 10 to about 65 minutes, preferably from about 20 to about 45 minutes, particularly from about 20 to about 35 minutes.

With respect to other preferred embodiments of the method as contemplated herein, the statements made regarding the agents and multi-component package units apply as contemplated herein apply mutatis mutandis.

### Examples

### 1.1. Coloration

The following formulations were produced (all data in wt. %):

Dye cream (F1)

| Raw material | wt. % |
|---|---|
| Cetearyl alcohol | 6.6 |
| C12-C18 fatty alcohols | 2.4 |
| Ceteareth-20 | 0.6 |
| Ceteareth-12 | 0.6 |
| Plantacare 1200 UP (laurylglucoside, 50-53% hydrous solution) | 2.0 |
| Sodium laureth-6 carboxylate (21% hydrous solution) | 10.0 |
| Sodium myreth sulfate (68-73% hydrous solution) | 2.8 |
| Sodium acrylate, trimethylammoniopropylacrylamide chloride | 3.8 |
| copolymer (19-21% hydrous solution) | |
| Sodium hydroxide | 0.26 |
| p-toluylendiamine, sulfate | 0.48 |
| m-aminophenol | 0.02 |
| 4-chlorresorcin | 0.09 |
| 2-methyl resorcin | 0.04 |
| Resorcin | 0.12 |
| Ammonium sulfate | 0.71 |
| Sodium sulfate | 0.4 |
| Ascorbic acid | 0.1 |
| 1-hydroxyethane-1,1-diphosphonic acid (60% hydrous solution) | 0.2 |
| Sodium soluble glass | 0.5 |
| L-serin | 1.0 |
| Ammonia (25% hydrous solution) | 6.7 |
| Water | ad 100 |

Oxidant (Ox)

| Raw material | wt. % |
|---|---|
| Sodium benzoate | 0.04 |
| Dipicolinic acid | 0.1 |
| Di-sodium pyrophosphate | 0.1 |
| Potassium hydroxide | 0.09 |
| 1,2-propylene glycol | 1.0 |
| 1-hydroxyethane-1,1-diphosphonic acid (60% hydrous solution) | 0.25 |
| Paraffinum liquidum | 0.30 |
| Steartrimonium chloride | 0.39 |
| Cetearyl alcohol | 3.4 |
| Ceteareth-20 | 1.0 |
| Hydrogen peroxide (50% hydrous solution) | 12.0 |

The dye cream (F1) and the oxidant (Ox) were then mixed together in a ratio of 1:1 and applied to the hair strands (Kerling Euro natural hair, white). The weight ratio of the application mixture: Hair 4:1, exposure time 30 minutes at a temperature of 32 degrees Celsius. The strands were then rinsed with water, dried and left to rest at room temperature for at least 24 hours. The hair strands were then measured colorimetrically.

The dye (F1+OX) colored the strands with a hazelnut brown tint.

1.2. Decolorizing (Multi-Component Package Unit with 2 Components)
Containers (1) and (2) were filled with the following agents (all specifications in grams):

Container (1) containing 20 g of agent (1)

| | Agent (1V) | | Agent (1E) | |
|---|---|---|---|---|
| Versagel M 1600⁽¹⁾ | 3.58 | g | 3.58 | g |
| Formamidine sulfinic acid | 8.00 | g | 8.00 | g |
| (2R,3R)-1,4-dimercapto-2,3-butanediol | — | | 2.00 | g |
| (CAS no. 3483-12-3) | | | | |
| Paraffinum liquidum | ad 20.00 | g | ad 20.00 | g |

⁽¹⁾Versagel M 2600: INCI Paraffinum liquidum (mineral oil), ethylene/propylene/styrene-copolymer, butylene/ethylene/styrene-copolymer

Container (2) containing 80 g of agent (2)

| Agent (2) | Agent (2) | |
|---|---|---|
| Cetearyl alcohol | 4.0 | g |
| PEG-40 hydrogenated Castor Oil | 0.80 | g |
| Sodium laureth-sulfate (C₁₂-C₁₄, 2 EO) | 0.54 | g |
| Monoethanolamine | 2.1 | g |
| Dipicolinic acid | 0.032 | g |
| 1-hydroxy ethane-1,1 diphosphonic | 0.12 | g |
| acid | | |
| Water | ad 80 | g |

20 g of agent (1) were mixed with 80 g of agent (2) by stirring at room temperature to produce the ready-to-use decolorizing agent. The pH value of the ready-to-use decolorizing agent was approximately 9.5.

Once the reducing agents in the ready-to-use decolorizing agent (agent (1) plus (2)) were dissolved completely, the ready-to-use decolorizing agent was applied to the previously dyed hair, left there for an exposure time x and then rinsed off. Then the hair was dried.

The coloring on the decolorized strands was visually assessed. The evaluation of the color-intensity took place based on the following scale:

0—Strands no longer have any perceptible color (white-blond, like the original color of the Kerling Euro natural hair, white)
1—Strands colored with mild color intensity
2—Strands colored with moderate color intensity
3—Strands colored with strong color intensity
4—Color of strands like immediately after the dyeing, no decolorizing effect

| | Ready-to-use decolorizing agent (2 components) | |
|---|---|---|
| | Agent (1V) + agent (2) | Agent (1E) + agent (2) |
| Application time x | Comparison | Present Disclosure |
| 15 minutes | 4 | 3 |
| 30 minutes | 4 | 2 |
| 45 minutes | 3 | 2 |
| 60 minutes | 2 | 1 |

1.3 Decolorizing (Multi-Component Package Unit with 3 Components)
Containers (1), (2) and (3) were filled with the following agents (all specifications in grams):

Container (1) containing 20 g of agent (1)

| | Agent (1) | |
|---|---|---|
| Versagel M 1600⁽¹⁾ | 3.58 | g |
| Formamidine sulfinic acid | 8.00 | g |
| Paraffinum liquidum | ad 20.00 | g |

⁽¹⁾Versagel M 2600: INCI Paraffinum liquidum (mineral oil), ethylene/propylene/styrene-copolymer, butylene/ethylene/styrene-copolymer

Container (2) containing 40 g of agent (2)

| | Agent (2V) | | Agent (2E) | |
|---|---|---|---|---|
| | Comparison | | Present Disclosure | |
| Cetearyl alcohol | 4.0 | g | 4.0 | g |
| PEG-40 hydrogenated Castor Oil | 0.80 | g | 0.80 | g |
| Sodium laureth-sulfate | 0.54 | g | 0.54 | g |
| (C₁₂-C₁₄, 2 EO) | | | | |
| (2R,3R)-1,4-dimercapto- | | — | 2.00 | g |
| 2,3-butanediol | | | | |
| (CAS no. 3483-12-3) | | | | |
| Dipicolinic acid | 0.032 | g | 0.032 | g |
| 1-hydroxy ethane-1,1 diphosphonic | 0.12 | g | 0.12 | g |
| acid | | | | |
| Water | ad 40.00 | g | ad 40.00 | g |

Container (3) containing 40 g of agent (3).

| | Agent (3) | |
|---|---|---|
| Cetearyl alcohol | 2.00 | g |
| Castor oil hydrogenated, 40 EO | 0.40 | g |
| Sodium laureth-sulfate (C₁₂-C₁₄, 2 EO) | 0.27 | g |
| Monoethanolamine | 2.1 | g |
| Water | ad 40.00 | g |

20 g of agent (1) were mixed with 40 g of agent (2) and 40 g of agent (3) by stirring at room temperature to produce the ready-to-use decolorizing agent. The pH value of the ready-to-use decolorizing agent was approximately 9.5.

Once the reducing agent (formamidine sulfinic acid) in the ready-to-use decolorizing agent (agent (1) plus (2) plus (3)) has dissolved completely, the ready-to-use decolorizing agent was applied to the dyed hair, left there for the exposure period x at room temperature and then rinsed off. Then the hair was dried.

The coloring on the decolorized strands was visually assessed. The evaluation of the color-intensity took place based on the following scale:

0—Strands no longer have any perceptible color (white-blond, like the original color of the Kerling Euro natural hair, white)
1—Strands colored with mild color intensity
2—Strands colored with moderate color intensity
3—Strands colored with strong color intensity
4—Color of strands like immediately after the dyeing, no decolorizing effect

| | Ready-to-use decolorizing agent (3 components) | |
|---|---|---|
| | Agent (1) + agent | |
| | (2V) + agent | Agent (1) + agent (2E) + agent |
| Application time x | (3) Comparison | (3) Present Disclosure |
| 15 minutes | 4 | 3 |
| 30 minutes | 4 | 2 |
| 45 minutes | 3 | 2 |
| 60 minutes | 2 | 1 |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Agent for the reductive decolorization of dyed keratinous fibers comprising, in a cosmetic carrier,
(a) formamidine sulfinic acid and/or salts thereof and
(b) at least one compound of the formula (I) 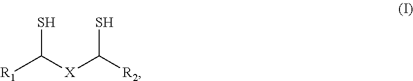
where
R1, R2 denote independently of one another a hydrogen atom, a carboxy group (—COOH) or the salt thereof, a C₁-C₆ alkyl group, a hydroxy C₁-C₆ alkyl group, or a C₁-C₆ alkoxy-carbonyl group,
X denotes a bivalent organic radical of formula (II) or a direct bond, 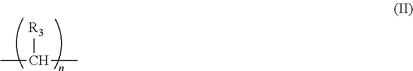
R3 denotes a hydroxy group, a hydrogen atom, a carboxy group
(—COOH) or the salt thereof, a C₁-C₆ alkoxy-carbonyl group, a C₁-C₆-alkyl group or an amino group, and
n denotes an integer from about 1 to about 4.

2. Agent according to claim 1 comprising—relative to the total weight of the agent—(a) formamidine sulfinic acid and/or salts thereof in a total amount of from about 0.1 to about 30.0 wt. %.

3. Agent according to claim 1 comprising (b) at least one compound of the formula (I), wherein
R1, R2 denote, independently from one another, a hydrogen atom, a carboxy group (—COOH) or the salt thereof.

4. Agent according to claim 1 comprising (b) at least one compound of the formula (I), wherein
X denotes a bivalent organic radical of formula (II), 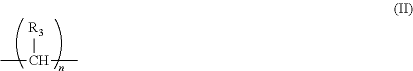
and
R3 denotes a hydroxy group or a hydrogen atom.

5. Agent according to claim 1 comprising (b) at least one compound of the formula (I), wherein
X denotes a bivalent organic radical of formula (II), 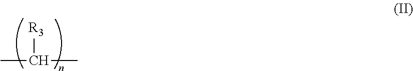
and
n denotes the number 2 or 3.

6. Agent according to claim 1 comprising (b) at least one compound of formula (I), which is selected from the group of propan-1,3-dithiol, 2-methylpropan-1,3-dithiol, 1,3-disulfanylpropan-2-ol, butan-1,4-dithiol, 2,3-dimethyl-butan-1,4-dithiol, 1,4-disulfanylbutan-2,3-diol (1,4-dimercapto-2,3-butanediol, dithiothreitol, dithioerythritol), pentan-1,5-dithol, 2,3,4-trimethyl-pentan-1,5-dithiol, 1,5-disulfanylpentan-2,3,4-triol, hexan-1,6-dithiol, 2,3,4,5-tetramethylhexan-1,6-dithiol, 1,6-disulfanylhexan-2,3,4,5-tetrol, ethan-1,2-dihiol, butan-2,3-dithiol, 2,3-disulfanylbutan-1,4-diol, 2,3-disulfanyl-1,4-butane dioic acid, 1,4-dimethyl-2,3-disulfanylbutandioate, 1,4-diethyl-2,3-disulfanylbutandioate and/or 3,4-disulfanylhexan-2,5-diol.

7. Agent according to claim 1, comprising—relative to the total weight thereof—(b) one or multiple compounds of formula (I) in a total quantity of from about 0.1 to about 30.0 wt. %.

8. Agent according to claim 1 wherein the weight ratio of (a) the total amount of all formamidine sulfinic acid (salts) to (b) the total amount of all compounds of formula (I) contained in the agent has a value of from about 1 to about 10.

9. Agent according to claim 1 further comprising a surfactant chosen from the group of anionic, amphoteric, zwitterionic, nonionic and/or cationic surfactants.

10. Agent according to claim 1 having a pH value in the range of from about 7.5 to about 12.5.

11. Multi-component package unit (kit-of-parts) for reductive decolorizing of keratinous fibers comprising, separately packaged:
a first container (1) comprising a cosmetic agent (1) and
a second container (2) comprising a cosmetic agent (2), wherein
agent (1) in container (1) comprises
(a) Formamidine sulfinic acid and/or salts thereof and
(b) At least one compound of formula (I), 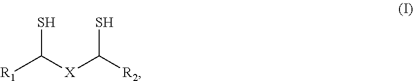
where
R1, R2 denote independently of one another a hydrogen atom, a carboxy group (—COOH) or the salt thereof, a C₁-C₆ alkyl group, a hydroxy C₁-C₆ alkyl group, or a C₁-C₆ alkoxy-carbonyl group,
X denotes a bivalent organic radical of formula (II) or a direct bond, 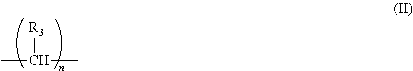
R3 denotes a hydroxy group, a hydrogen atom, a carboxy group (—COOH) or the salt thereof, a C₁-C₆ alkoxy-carbonyl group, a C₁-C₆-alkyl group or an amino group, and
n denotes an integer from about 1 to about 4,
and
agent (2) in container (2) comprises
(c) one or multiple alkalizing agents.

12. Multi-component package unit (kit-of-parts) for reductive decolorizing of keratinous fibers comprising, separately packaged:
a first container (1) comprising a cosmetic agent (1) and
a second container (2) comprising a cosmetic agent (2), wherein
agent (1) in container (1) comprises
(a) Formamidine sulfinic acid and/or salts thereof and
agent (2) in container (2) comprises
(b) At least one compound of the formula (I), 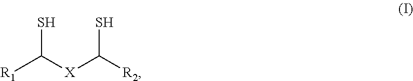
where
R1, R2 denote independently of one another a hydrogen atom, a carboxy group (—COOH) or the salt thereof, a C₁-C₆ alkyl group, a hydroxy C₁-C₆ alkyl group, or a C₁-C₆ alkoxy-carbonyl group,
X denotes a bivalent organic radical of formula (II) or a direct bond, 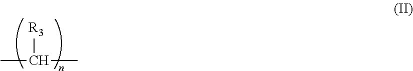
R3 denotes a hydroxy group, a hydrogen atom, a carboxy group (—COOH) or the salt thereof, a C₁-C₆ alkoxy-carbonyl group, a C₁-C₆-alkyl group or an amino group, and
n denotes an integer from about 1 to about 4.

13. Multi-component package unit according to claim 12, further comprising:
a third container (3) comprising a cosmetic agent (3), wherein
agent (3) in container (3) comprises
(c) one or multiple alkalizing agents.

14. Multi-component package unit according to claim 11, wherein alkalizing agent (c) is selected from the group of ammonia, 2-aminoethan-1-ol, 2-amino-2-methylpropan-1-ol, arginine, lysine, ornithine, histidine, potassium hydroxide, sodium hydroxide, magnesium hydroxide and/or calcium hydroxide.

15. (canceled)

16. (canceled)

17. Agent according to claim 1 comprising—relative to the total weight of the agent—(a) formamidine sulfinic acid and/or salts thereof in a total amount of from about 6.0 to about 10.0 wt. %.

18. Agent according to claim 1 comprising (a) at least one compound of the formula (I), wherein
X denotes a bivalent organic radical of formula (II), 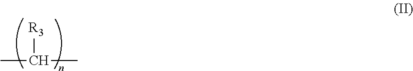
and
n denotes the number 2.

19. Agent according to claim 1 comprising—relative to the total weight thereof—(b) one or multiple compounds of formula (I) in a total quantity of from about 0.5 to about 6.0 wt. %, wherein the weight ratio of (a) the total amount of all formamidine sulfinic acid (salts) to (b) the total amount of all compounds of formula (I) contained in the agent has a value of from about 3 to about 6.

20. Agent according to claim 1 having a pH value in the range of from about 8.5 to about 10.0.

21. Agent according to claim 1 comprising—relative to the total weight thereof—(b) one or multiple compounds of formula (I) in a total quantity of from about 0.1 to about 30.0 wt. %, wherein
R1, R2 denote, independently from one another, a hydrogen atom, a carboxy group (—COOH) or the salt thereof,
X denotes a bivalent organic radical of formula (II), 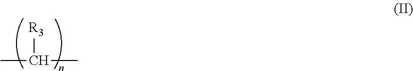
R3 denotes a hydroxy group or a hydrogen atom
n denotes the number 2 or 3, and
the weight ratio of (a) the total amount of all formamidine sulfinic acid (salts) to (b) the total amount of all compounds of formula (I) contained in the agent has a value of from about 1 to about 10.

22. Multi-component package unit according to claim 12, wherein agent (2) in container (2) further comprises
(c) one or multiple alkalizing agents.
